(19) United States
(12) Patent Application Publication
(10) Pub. No.: US 2016/0030530 A1
(43) Pub. Date: Feb. 4, 2016

(54) SULFORAPHANE/SULFORAPHANE PRECURSOR AND PHYTOSTEROL/PHYTOSTANOL COMPOSITIONS

(71) Applicant: NUTRAMAX LABORATORIES, INC., Edgewood, MD (US)

(72) Inventors: Brian CORNBLATT, Westminster, MD (US); Anton BZHELYANSKY, Baltimore, MD (US); Robert HENDERSON, Street, MD (US); Chia-Ping Charles HSU, Ellicott City, MD (US)

(21) Appl. No.: 14/775,089

(22) PCT Filed: Mar. 15, 2014
(86) PCT No.: PCT/US14/29976

§ 371 Date: Sep. 11, 2015

Related U.S. Application Data
(60) Provisional application No. 61/794,417, filed on Mar. 15, 2013.

(51) Int. Cl. A61K 38/47; A61K 31/375; A61K 45/06; A61K 36/31; A61K 31/275; A61K 31/7028; A61K 31/575

(52) U.S. Cl. CPC A61K 38/47; A61K 31/7028; A61K 31/375; A61K 31/575; A61K 36/31; A61K 31/275; A61K 45/06; C12Y 302/01147

(57) ABSTRACT

The invention relates to the combination of a sulforaphane precursor, an enzyme capable of converting the sulforaphane precursor to sulforaphane, an enzyme potentiator, and a phytosterol and/or phytostanol or ester thereof. The invention also relates to the combination of a sulforaphane or a derivative thereof and a phytosterol and/or phytostanol or ester thereof. The invention also relates to the combination of a broccoli extract or powder and a phytosterol and/or phytostanol or ester thereof. The invention provides compositions and methods relating to these combinations.

### CROSS-REFERENCE TO RELATED APPLICATION

This application claims priority to the U.S. Provisional Patent Application No. 61/794,417, filed on Mar. 15, 2013, which is incorporated by reference in its entirety.

### FIELD OF THE INVENTION

The present invention relates to the combination of a sulforaphane precursor, an enzyme capable of converting the sulforaphane precursor to sulforaphane, an enzyme potentiator, and a phytosterol and/or phytostanol or an ester thereof. The present invention also relates to the combination of a sulforaphane or a derivative thereof and a phytosterol, phytostanol or an ester thereof. The present invention also relates to the combination of a broccoli extract or powder and a phytosterol and/or phytostanol or ester thereof. The present invention provides compositions and methods relating to these combinations.

### BACKGROUND OF THE INVENTION

Connective tissue is the structural framework of cartilage, bone, synovium, ligament, meniscus, and tendon in articulating joints. Components of connective tissue are produced by resident cells and then secreted to form the extracellular matrix (ECM) characteristic of the tissue. In addition to serving as structural framework, the ECM also plays a critical role in cell communication and function. In articular cartilage, chondrocytes are aligned in a distinct pattern within the type II collagen ECM framework. Bone forming osteoblasts and osteocytes, as well as bone resorbing osteoclasts, are organized in mineralized type I collagen ECM. The few fibroblast-like and macrophage-like cells in the synovium are also held in place by ECM. Similarly, tenocytes and ligament cells are assembled together within the ECM. The synthesis and breakdown of connective tissue ECM is controlled by a network of regulatory molecules which are also produced by the resident tissue cells. This network includes growth factors and a wide array of molecules known as pro-inflammatory mediators. They include cytokines, chemokines, prostaglandins and nitric oxide. These molecules exhibit many biological activities. They can induce cell proliferation or cell death. These substances can also induce anabolic pathways for production of ECM or induce catabolic enzymes that can break down the ECM. Under physiological conditions, cell survival or death, the production or breakdown of connective tissue ECM is tightly controlled to maintain balanced homeostasis. The production and function of regulatory molecules is modulated by many factors including mechanical forces, physical factors such as temperature and pH, chemicals, microbes and their products. Under certain conditions, these factors can elicit excessive and untimely production of regulatory molecules leading to irreparable tissue damage, loss of function and death.

Tissues react to mechanical, physical, chemical insults and infection by an inflammatory response. The inflammation process is known to lead to recovery, to healing, defense against infection and is usually life preserving. The inflammatory response in humans and animals consists of two phases. The initial phase is characterized by the local synthesis of pro-inflammatory mediators such prostaglandins and leukotrienes. They are derived from arachidonic acid through the action of cyclooxygenases and lipoxygenases. These pro-inflammatory mediators increase local blood flow and enhance the permeability of endothelial cells to allow leukocyte recruitment and accumulation. Other pro-inflammatory mediators which are subsequently produced include cytokines (IL-1β, TNF-α), chemokines (IL-8), and nitric oxide. In the second phase, the resolution phase, prostaglandins generated during the initial phase activate enzymatic pathways along which arachidonic acid is converted to chemical mediators with anti-inflammatory properties. It has been reported that prostaglandin E₂ (PGE₂) activates the expression of 15-lipoxygenase which generates anti-inflammatory lipoxins from arachidonic acid. Thus, the resolution of inflammation is driven by the pro-inflammatory response. Studies have revealed that the initiation, progression and termination of the inflammation process are tightly controlled. Prolonged, exaggerated inflammation has been associated with many disorders including osteoarthritis (OA), rheumatoid arthritis (RA), Alzheimer's disease, cardiovascular disease, and even cancer.

In joint tissues, chondrocytes, synoviocytes, osteoblasts, osteoclasts, ligament cells, and tenocytes produce a wide array of pro-inflammatory mediators. Among these is prostaglandin E2 (PGE2), which is known to play a regulatory role by inducing the production of other mediators including cytokines, nitric oxide, and connective tissue degrading matrix metalloproteinase (MMP) enzymes. Due to its ability to induce MMPs, PGE2 contributes to the breakdown of cartilage ECM. In addition, PGE2 promotes bone resorption and osteophyte formation. PGE2 sensitizes nociceptors on peripheral nerve endings, thereby contributing to the development of inflammatory pain. PGE2 levels are locally regulated by the inducible cyclooxygenase-2 (COX-2) enzyme, a nitric oxide synthase in chondrocytes that inhibits cartilage and proteoglycan degradation. In pathologic conditions such as osteoarthritis, COX-2 expression is up-regulated with a concomitant increase in PGE2 production.

The role of other tissues in the inflammation process is also well established. Inflammation of the synovial membrane is now recognized to be a key event in cartilage degradation in osteoarthritis, particularly during the early stages of the disease. Synovitis is characterized by activation of resident macrophage-like cells and fibroblast-like cells in the synovial membrane which leads to production of excessive amounts of pro-inflammatory mediators including TNF-alpha, IL-1 beta, and PGE2. Recent evidence suggests that synovial macrophages are the main source of the cytokines in the earliest stages of osteoarthritis and that they are important contributors to the cartilage damage in osteoarthritis throughout the course of the disease. Cytokines also induce production of PGE2 and active MMPs. It is now well accepted that these mediators control the balance between ECM destruction and repair, which has made these molecules preferred targets for therapeutic intervention. Other tissues in the joint such as the subchondral bone also produce pro-inflammatory mediators that modulate joint health.

In addition to pro-inflammatory mediators such as cytokines and prostaglandins, reactive oxygen species (ROS) have also been implicated in joint degeneration observed in osteoarthritis. Oxidative stress induced by ROS such as nitric oxide and hydrogen peroxide has been shown to cause chondrocyte apoptosis and cartilage ECM breakdown. Moreover, ROS have been reported to activate signal transduction pathways that lead to an increased production of pro-inflammatory mediators including cytokines and prostaglandins. Studies in vitro have demonstrated a linkage between the pathways involved in the production of ROS and pro-inflammatory mediators. These studies support the notion that agents capable of inhibiting both oxidative stress and inflammation pathways would be particularly useful in the modulation of inflammation.

The central role of COX-2 and PGE2 in the pathophysiology of osteoarthritis is reflected in the widespread use of selective COX-2 inhibitors and a variety of non-selective non-steroidal anti-inflammatory drugs (NSAIDs) for the treatment of the disorder. However, prolonged administration of these drugs has adverse side effects, including gastrointestinal pathologies and disruption of cartilage proteoglycan metabolism. Studies in human and animal models have demonstrated impaired bone healing and repair with the use of COX inhibitors. Therefore, there is a need for alternative treatments for the management of inflammation that do not center on the use of NSAIDs to inhibit the production of PGE2 and other pro-inflammatory mediators.

The use of natural products is becoming increasingly popular with humans and companion animals. Many of these products can be useful as chemoprotective agents, and many are useful in cardiovascular health and/or joint health, in particular, in the management of inflammation. Some natural products are being incorporated into dietary supplements, nutraceuticals, and medical foods.

Chemoprotection through the use of natural products is evolving as a safe, effective, inexpensive, easily accessible, and practical means to prevent or reduce the occurrence of many conditions affecting humans and domesticated animals. It is known that carcinogens which can damage cells at the molecular level are often ingested and inhaled as non-toxic precursors. These non-toxic precursors may then convert into carcinogenic substances in the body. Chemoprotective agents, such as natural substances which can activate detoxifying enzymes or their co-factors, can counteract and allow for the elimination of carcinogens. These same natural substances can potentiate other naturally existing defenses such as the immune system.

Some natural products have antioxidant activity. Oxidative stress plays a major role in aging, the progression of neurodegenerative diseases as well as physiological trauma, such as ischemia. Antioxidant agents can reduce or inhibit the oxidation of vital biomolecules and may play a role in treating, preventing, or reducing the occurrence of cancer, coronary heart disease, stroke, and neurodegenerative diseases. Alzheimer's Disease, dementia, and stroke are examples of conditions affected by oxidative stress.

An example of a natural product thought to have chemoprotective and antioxidant properties is sulforaphane. Sulforaphane is an organosulfur compound which is also known as 1-isothiocyanato-4-methylsulfinylbutane. The sulforaphane precursor, glucoraphanin, can be obtained from vegetables of the Brassicaceae family, such as broccoli, brussels sprout, and cabbage. However, copious amounts of vegetables must be consumed in order to obtain levels adequate for chemoprevention. Glucoraphanin is converted into sulforaphane by a thioglucosidase enzyme called myrosinase, which occurs in a variety of exogenous sources such as Brassicaceae vegetables and endogenously in the gut microflora. However, upon ingestion of glucoraphanin, not all animals are capable of achieving its conversion to sulforaphane, most likely due to variations in microflora populations and overall health. In addition, in acidic environments such as the stomach, glucoraphanin can be converted to inert metabolites. The active metabolite, sulforaphane is able to induce nuclear factor erythroid-2-related factor (Nrf2) which, in turn, upregulates the production of Phase II detoxification enzymes and cytoprotective enzymes such as glutathione S-transferases, NAD(P)H:quinine oxidoreductase (NQO1), and heme-oxygenase-1 (HO-1). Sulforaphane has been thought to induce the production of these enzymes without significantly changing the synthesis of P-450 cytochrome enzymes. The upregulation of Phase II enzymes is thought to play a role in a variety of biological activities, including the protection of the brain from cytotoxicity, the protection of the liver from the toxic effects of fat accumulation, and the detoxification of a variety of other tissues.

Sulforaphane and its precursor glucoraphanin have been studied extensively. Shapiro et al. (Nutrition and Cancer, (2006), Vol. 55(1), pp. 53-62) discusses a clinical Phase I study determining the safety, tolerability, and metabolism of broccoli sprout glucosinolates and isothiocyanates. Shapiro et al. discusses a placebo-controlled, double-blind, randomized clinical study of sprout extracts containing either glucosinolates such as glucoraphanin or isothiocyanates such as sulforaphane in healthy human subjects. The study found that administration of these substances did not result in systematic, clinically significant, adverse effects.

Phytosterols and phytostanols, which are also sometimes referred to as plants sterols and stanols, are a group of compounds which are typically found in plants. Phytosterols and phytostanols are structurally similar to cholesterol but differ in the structure of the side chain. Both phytosterols and phytostanols typically consist of a steroid skeleton with a hydroxyl group attached to the C-3 atom of the A ring and an aliphatic side chain attached to the C-17 atom of the D ring. Phytosterols have a double bond, typically between the C-5 and C-6 of the sterol moiety. In phytostanols, this bond is saturated.

Phytosterols and phytostanols have been known to have beneficial health effects. For example, phytosterols and phytostanols have been thought to be effective in lowering serum cholesterol levels, in particular total cholesterol and LDL cholesterol levels. Although the mechanism of action relating to the cholesterol-lowering effect is not fully understood, phytosterols and phytostanols are thought to be effective in reducing the absorption of cholesterol from the digestive tract.

Phytosterols and phytostanols have also been known to have beneficial immune-modulating properties. Bouic et al. (“Plant Sterols and Sterolins: A Review of Their Immune-Modulating Properties,” Alternative Medicine Review, 1999, Vol. 4(3), pp. 170-177) discusses a study assessing the protective effect of beta-sitosterol (BSS) and its glycoside (beta-sitosterol glycoside, or BSSG). In particular, the study showed that a mixture of BSS and BSSG exhibited anti-inflammatory, anti-neoplastic, anti-pyretic, and immune-modulating activity, possibly through its activity in targeting specific T-helper lymphocytes (T_{H}1 and T_{H}2 cells) to help normalize their functioning, which can result in improved T-lymphocyte and natural killer cell activity. BSS and BSSG was also thought to have a dampening effect on overactive antibody responses, as well as normalization of the DHEA:cortisol ratio and decline in interleukin-6 (IL-6) serum levels. Gabay et al. (“Stigmasterol: a phytosterol with potential anti-osteoarthritic properties,” Osteoarthritis and Cartilage, 2010, Vol. 18, pp. 106-116) discusses a study on the effect of stigmasterol on inflammatory mediators and metalloproteinases produced by chondrocytes. The study showed that stigmasterol inhibits several pro-inflammatory and matrix degradation mediators typically involved in osteoarthritis-induced cartilage degradation, such as MMP-3, MMP-13, ADAMTS-4, and PGE₂ at least in part through counteracting IL-1β-induced NF-κB pathway.

More than 200 phytosterols and related compounds have been identified. Examples of phytosterols and phytostanols include, but are not limited to: sitosterol (3β-stigmast-5-en-3-ol, CAS number 83-46-5), sitostanol (3β,5α-stigmastan-3-ol, CAS number 83-45-4), campesterol (3β-ergost-5-en-3-ol, CAS number 474-62-4), campestanol (3β,5α-ergostan-3-ol, CAS number 474-60-2), stigmasterol (3β-stigmasta-5,22,-dien-3-ol, CAS number 83-48-7), and brassicasterol (3β-ergosta-5,22,-dien-3-ol, CAS number 474-67-9).

For use in commercial products, phytosterols are typically isolated from vegetable oils, such as soybean oil, rapeseed (canola) oil, safflower oil, cottonseed oil, sunflower oil or corn oil, or from “tall oil,” which is a by-product of the manufacture of wood pulp. Phytosterols are then typically hydrogenated to obtain phytostanols. Free phytosterols and phytostanols are typically high melting powders which are insoluble in water, relatively soluble in oil, and soluble in alcohols. Both phytosterols and phytostanols can be esterified with fatty acids, for example, of vegetable oil origin, and the resulting esters are liquid or semi-liquid materials. Phytosterol esters and phytostanol esters are thought to generally have comparable chemical and physical properties to edible fats and oils, and therefore, supplementation of various processed foods with phytosterol ester and phytostanol esters is enabled. Phyosterols, phytostanols, and their esters and methods of making esters are described in U.S. Pat. No. 5,892,068; U.S. Pat. No. 7,771,771; U.S. Patent App. Pub. No. 2003/0104035; U.S. Pat. No. 8,338,564, and Cantrill et al. Phytosterols, Phytostanols and their Esters (CTA) 2008, each of which are incorporated by reference in their entirety.

Additional components are thought to have some beneficial effects for joint health and inflammation. Glucosamine is an example of an aminosugar, and it, is naturally formed in the body from glucose. When supplied exogenously, glucosamine stimulates connective tissue cell synthesis, increasing the amounts of normal extracellular matrix. Glucosamine is also the building block for glycosaminoglycans (“GAGs”) in cartilage and other connective tissues, thus, supplying additional glucosamine supplies the body with extra raw materials for matrix synthesis in connective tissues. Aminosugars may be natural, synthetic or semi-synthetically derived. Salts of glucosamine include but are not limited to glucosamine hydrochloride and glucosamine sulfate, glucosamine phosphate. Mannosamine and N-acetylglucosamine are other examples of aminosugars. Aminosugars can be chemically modified by, for example, esterification, sulfation, polysulfation, acetylation, and methylation.

Chondroitin is an example of a glycosaminoglycan (GAG) as described. Chondroitin sulfate is the most abundant glycosaminoglycan in articular cartilage and is also present in many other connective tissues in the body. Additionally, chondroitin sulfate competitively inhibits degradative enzymes that degrade connective tissues under conditions of abnormal, excessive inflammation. Chondroitin sulfate is a polymer composed of repeating units of glucuronic acid and sulfated galactosamine. [Lester M. Morrison, M. D. and O. Arne Schjeide, Ph.D., Coronary Heart Disease and the Mucopolysaccharides (Glycosaminoglycans) 12 (1974); Philip C. Champe and Richard A. Harvey, Lippincott's Illustrated Reviews: Biochemistry, 148-50 (2^{nd} ed. 1994)].

Avocado/soybean unsaponifiables (ASU) have been used to treat osteoarthritis and other forms of arthritis [Thiers, M. H., “Unsaponifiable constituents of avocado and soya oils. Treatment of certain forms of arthralgia,” J. Med. Lyon 53(222):195-8 (February 1972) (article in French)], as well as soft-tissue inflammatory conditions [Trevoux, R., “Unsaponifiable fractions of the avocado and soybean in gynecology,” J. Bynecol. Obstet. Biol. Reprod. 6(1):99-105 (January 1977) (article in French); Lamaud, M. E., et al., “Biochemical modifications of connective tissue induced by the non-saponifiables of avocado and soy-bean oils administered percutaneously in the ‘hairless’ rat,” Pathol. Biol. 26(5):269-74 (May-June 1978) (article in French)]. The mechanism of action of this compound is to stimulate chondrocyte expression of TGF (transforming growth factor) beta 1, TGF beta 2 and plasminogen activator inhibitor 1 (“PAI-1”). By increasing PAI-1, ASU blocks the cascade that leads to metalloproteinase activation [Boumediene K., et al., “Avocado/soya unsaponifiables enhance the expression of transforming growth factor beta 1 and beta 2 in cultured articular chondrocytes,” Arthritis Rheum. 42(1): 148-56 (January 1999)]. ASU mixtures also thought to reduce the spontaneous production of stromelysins, IL-6, IL-8 and prostaglandin E2 by chondrocytes. Additionally, ASU may decrease the effects of IL-1, and thereby reduce chondrocyte and synoviocyte production of collagenase. [Henrotin, Y. E., et al., “Effects of three avocado/soybean unsaponifiable mixtures on metalloproteinases, cytokines and prostaglandin E2 production by human articular chondrocytes,” Clin. Rheumatol. 17(1): 31-9 (1998).]

The gum resin of Boswellia serrata, a traditional Ayurvedic medicine) contains two boswellic acids, 11-keto-β-boswellic acid (KBA) and acetyl-11-keto-b-boswellic acid (AKBA). Abdel-Tawab, et al. report that b-boswellic acids inhibit the inflammatory-related enzymes microsomal prostaglandin E synthase-1 and serine protease cathepsin G, thereby showing these boswellic acids have anti-inflammatory characteristics and would be well suited as constituents in joint health nutraceuticals [Clin Pharmacokinet 2011; 50 (6): 349-369]

Green tea contains a mixture of catechins, including epicatechin (EC), epigallocatechin (EGC), epicatechin gallate (ECG) and epigallocatechin gallate (EGCG). These catechins have potent antioxidant activity, acting as scavengers of the free radicals (ROS and RNS) involved in damage to cells. They also act by chelating metals that catalyze production of ROS (1). This antioxidant activity may interfere with the damaging effects of agents, e.g. fibronectin fragments (Fn-f) and cytokines, that can cause DJD. Antioxidants block the effects of Fn-f, which include increased expression and activity of both cytokines IL-1 and TNF-a (2,3). In addition, recent studies have shown that green tea polyphenols significantly reduce the incidence of collagen-induced arthritis in mice that was associated with reduced expression of TNF-a and cyclooxygenase 2, a TNF-a regulated enzyme that catalyzes the production of prostaglandin E2 (4). Other studies have shown that the EGCG in green tea inhibits IL-1 induced expression of nitric oxide synthase and nitric oxide production and suppresses activation of nuclear factor-kB, a key step in initiation of the cytokine effects (5). Furthermore, the catechins in green tea were recently shown to potently inhibit aggrecanase activities known to be involved in the early stages of destruction of cartilage proteoglycans (6). Components of green tea have the potential to ameliorate the cause and the symptoms of DJD through multiple mechanisms. The green tea may be administered as an extract or standardized to polyphenols or catechins.

Methylsulfonylmethane (MSM), also known as DMSO₂, methyl sulfone, and dimethyl sulfone, is an organosulfur compound. MSM is thought to provide sulfur which is potentially used by proteins to form disulfide bonds. GAGs use sulfur to cross-link together via these disulfide bonds. These bonds reduce conformational flexibility of GAG chains, making cartilage firm and resilient.

Hyaluronic acid (HA) is a high molecular weight polysaccharide that is distributed in all bodily tissues and fluids and is one constituent of the extracellular matrix of articular cartilage. The viscoelastic properties of HA play a critical role in joint mechanics in synovial fluid. When HA is bound to aggrecan, large negatively charged aggregates form which attract water molecules which help to cushion the joint. Exogenous HA has anti-inflammatory, anabolic, analgesic, and chondroprotective effects [Sports Health. March 2013; 5(2): 153-159].

Lipoic acid (LA), also known as 1,2 dithiolane-3-pentanoic acid, 1,2-dithiolane-3-valeric acid, or 6,8-thioctic acid, is a potent, naturally occurring, low molecular weight antioxidant. Lipoic acid is synthesized enzymatically in the mitochondrion from octanoic acid. It is a critical cofactor of mitochondrial decarboxylation reactions and is essential for adequate ATP production. Lipoic acid exists in enantiomeric forms: R-lipoic acid (R-LA) and S-lipoic acid (S-LA). In biological systems, only R-LA is conjugated to lysine residues in the amide linkage. The oxidized (LA) and reduced (DHLA) forms represent a potent redox couple. The biological effect of LA include scavenging of reactive oxygen species, regeneration of endogenous antioxidants such as glutathione and vitamin E, metal ion chelating, and repair oxidative damage in macromolecules. Both LA and DHLA are capable of scavenging reactive oxygen species (ROS) and reactive nitrogen species (RNS), and have the ability to prevent protein carbonyl formation. LA and DHLA can regenerate other endogenous antioxidants such as vitamin C, vitamin E, and glutathione, thereby protecting cells against oxidative stress. Recent evidence suggests that LA not only acts as a true oxidant scavenger but in addition acts as an activator of cellular stress response pathways. Derivatives of lipoic acid have been described in the art. Some derivatives of lipoic acid provide improved biological activity, improved pharmacokinetic properties such as longer half lives, improved bioavailability, and decreased drug interaction profiles. Derivatives of lipoic acid have been described in the following publications, hereby incorporated by reference: Gruzman et al. Synthesis and characterization of new and potent alpha-lipoic acid derivatives. Bioorganic & Medicinal Chemistry, 2004, 12:1183-1190; Melagraki et al. Synthesis and evaluation of the antioxidant and anti-inflammatory activity of novel coumarin-3-aminoamides and their alpha-lipoic acid adducts. European Journal of Medicinal Chemistry, 2009, 44:3020-3026; Gurkan et al., Syntheses of novel indole lipoic acid derivatives and their antioxidant effects on lipid peroxidation. Archiv der Pharmazie, 2005, 338:67-73; Ortial et al., Fluorinated amphiphilic amino acid derivatives as antioxidant carriers: a new class of protective agents. J Med Chem 2006; 12-2820; and Koufaki et al. Sign and synthesis of antioxidant alpha-lipoic acid hybrids. Methods Mol Biol, 2010, 594:297-309.

Boron is a non-metallic element that is found naturally in the environment. Boron supplementation has been shown to alleviate arthritic pain and discomfort. Epidemiological studies have uncovered analytical evidence of lower boron concentrations in femur heads, bones, and synovial fluid from people with arthritis compared to those without; in areas of the world where boron intakes usually are 1.0 mg or less/day the estimated incidence of arthritis ranges from 20 to 70%, whereas in areas of the world where boron intakes are usually 3 to 10 mg, the estimated incidence of arthritis ranges from 0 to 10% [Environ Health Perspect. 1994 November; 102 Suppl 7:83-5].

Collagen type II also has beneficial effects that help maintain the normal balance between anabolism and catabolism. Specifically, connective tissue diseases may result from autoimmune processes, in which the immune system attacks and catabolizes the individual's own connective tissues as if it were a “foreign invader.” Oral administration of collagen type II can desensitize the immune system, preventing further attack and normalizing immune responses in these individuals. This decreases catabolic processes in the connective tissues and maximize anabolism. Ingestion of collagen type II presents this molecule to the immune cells in the gut-associated lymphoid tissues (GALT, a.k.a., Peyer's patches). Interactions between the collagen molecule and specific cells within the GALT activate mobile immune cells called T suppressor cells. These cells, in turn, moderate the destructive immune reaction against the individual's own collagen type II (in connective tissues).

Resveratrol is a stilbenoid, commonly found in grapes and in the roots of the Japanese Knotweed during stress and bacterial or fungial infection. In mouse and rat experiments, resveratrol has been shown to play a role in telomere lengthening, telomerase activity enhancement, blood sugar-lowering, inhibition of platelet aggregation, promotion of vasodilation by enhancing the production of NO and have anti-inflammatory properties.

Gallic acid is a trihydroxybenzoic acid naturally occuring in gallnuts, sumac, witch hazel, tea leaves and oak bark. Studies have shown gallic acid to have anti-oxidative, pro-apoptopic and anti-inflammatory properties.

Omega-3 fatty acids are essential fatty acids including ALA, DHA and EPA that are not naturally produced in the body and therefore need to be consumed in the diet typically by eating fish. Studies demonstrate that Omega-3 fatty acids are effective at helping to lower triglycerides and blood pressure. Additional studies have shown Omega-3 fatty acids to have an anti-inflammatory effect in the vasculature and in joints.

Krill oil is rich in the omega-3 fatty acids eicosapentaenoic acid (EPA) and docosahexaenoic acid (DHA) and the anti-oxidant astaxanthin. Krill oil has been shown to possess anti-oxidant properties, lower cholesterol, and lower C-Reactive Protein, an inflammatory marker associated with increased risk of heart disease risk. Studies also reveal anti-inflammatory effects and reduced pain and stiffness associated with rheumatoid and osteoarthritis.

S-adenosylmethionine (SAMe) is an important endogenous compound, present throughout the body, and it takes part in a great number of biologic reactions such as transsulfation reactions. In this role it is an important reactant in the synthesis of many structural components of connective tissues, including proteins and proteoglycans. Thus, SAMe has significant anabolic effects which would enhance the actions of other anabolic agents. SAMe also has anti-inflammatory effects by virtue of its antioxidant action. The primary CNS function of SAMe is to donate methyl groups in the reactions synthesizing various crucial compounds, including neurotransmitters and phospholipids. For example, SAMe facilitates the conversion of phosphatidylethanolamine to phosphatidylcholine, which forms part of the inner, lipid layer of the plasma membrane. In so doing, SAMe increases membrane fluidity and enhances effectiveness of receptor/ligand binding. [Champe and Harvey, Biochemistry, 1994; Stramentinoli, G., “Pharmacologic Aspects of S-Adenosylmethionine,” American J. Med., 83(5A):35 (1987); Baldessarini, F., “Neuropharmacology of S-Adenosyl Methionine,” American J. Med., 83(5A):95 (1987); Carney, M., “Neuropharmacology of S-Adenosyl Methionine,” Clin. Neuropharmacol., 9(3):235 (1986); Janicak, P., “S-Adenosylmethionine in Depression,” Alabama J. Med. Sci. 25(3):306 (1988)]. These functions may also pertain to other methyl donors such as betaine (trimethylglycine), 5-methyltetrahydrofolate, folic acid, and dimethylglycine. [Champe and Harvey, Biochemistry, 1994].

Silymarin and the active components of silymarin have several mechanisms of action, including stimulation of nucleolar polymerase A. This stimulation in turn increases ribosomal activity leading to increased synthesis of cellular proteins, and an increased rate of hepatocellular repair. Conti, M., et al., Protective activity of Silipide on liver damage in rodents, Japan J. Pharmacol., 60, 1992, pp. 315-21. Other protective mechanisms involve changes in the molecular structure of the hepatocellular membrane, which reduce binding and entry of toxins into the cell, and an antioxidant effect. Parish, R. & Doering, P., Treatment of Amanita mushroom poisoning: a review, Vet. Hum. Toxocol., 28 (4) 1986, pp. 318-22.

Vitamin K2, which is also known as menaquinone, can be provided in the form of menaquinone-4 (MK-4), menaquinone-5 (MK-5), menaquinone-6 (MK-6), menaquinone-7 (MK-7), menaquinone-8 (MK-8), menaquinone-9 (MK-9), menaquinone-10 (MK-10), menaquinone-1 1 (MK-1 1), and phylloquinone. Phylloquinone can be obtained from plant sources such as green leafy vegetables and has a short half-life in the plasma, but it can be converted to menaquinone-4 (MK-4) by the endothelium, testes and pancreas. It can be synthesized by intestinal bacteria and is also found in cheeses.

European Patent Application No. 2 213 280 discloses formulations comprising glucosinolates such as glucoraphanin and myrosinase, wherein the formulation is encapsulated or coated.

All references cited herein are incorporated by reference in their entirety.

### SUMMARY OF THE INVENTION

The present invention provides a composition comprising: (i) a sulforaphane precursor, preferably glucoraphanin; (ii) an enzyme capable of converting the sulforaphane precursor to sulforaphane, preferably a glucosidase enzyme, more preferably a thioglucosidase enzyme, and most preferably myrosinase; (iii) an enzyme potentiator, preferably ascorbic acid; and (iv) a phytosterol and/or phytostanol or an ester thereof. The present invention also provides a composition comprising: (i) sulforaphane or a derivative thereof, and (ii) a phytosterol and/or phytostanol or an ester thereof. The present invention also provides a composition comprising: (i) a broccoli extract or powder, and (ii) a phytosterol and/or phytostanol or ester thereof.

The present invention also provides methods comprising administering one or more of the combinations described in the present application. The present invention provides a method of treating, preventing, reducing the occurrence of, decreasing the symptoms associated with, and/or reducing secondary recurrences of, a disease or condition or damage associated with the connective tissue, liver, prostate, brain, spine, lung, kidneys, colon, breast, esophagus, pancreas, or ovaries in a subject, comprising administering to a subject in need thereof one of the compositions of the present invention. The present invention further provides methods of treating, preventing, reducing the amount or degree, decreasing the symptoms associated with inflammation. The present invention also provides a method of decreasing levels of or downregulating or decreasing gene expression of matrix metalloproteinases such as matrix metalloproteinase 13 (MMP-13) in a subject, comprising administering to the subject thereof one of the compositions of the present invention. The present invention also provides a method of treating, preventing, reducing the occurrence of, decreasing the symptoms associated with, and/or reducing secondary recurrences of a condition or disorder associated with increased or abnormal levels of MMP-13 and/or PGE₂ in a subject in need thereof, comprising administering to the subject one of the compositions of the present invention.

### BRIEF DESCRIPTION OF THE FIGURES

FIG. 1 is a graph showing the conversion of glucoraphanin at 38° C. without ascorbic acid, as described in Example 4.

FIG. 2 is a graph showing the conversion within about 10 minutes at 38° C. as a function of ascorbic acid concentration, as described in Example 4.

FIG. 3 is a graph showing the conversion to sulforaphane within 30 minutes at 38° C. and 1 mM ascorbic acid, as described in Example 4.

FIG. 4 is a graph showing the conversion of glucoraphanin to sulforaphane in simulated intestinal fluid, as described in Example 5.

FIG. 5 is a graph showing the results of the experiment described in Example 6.

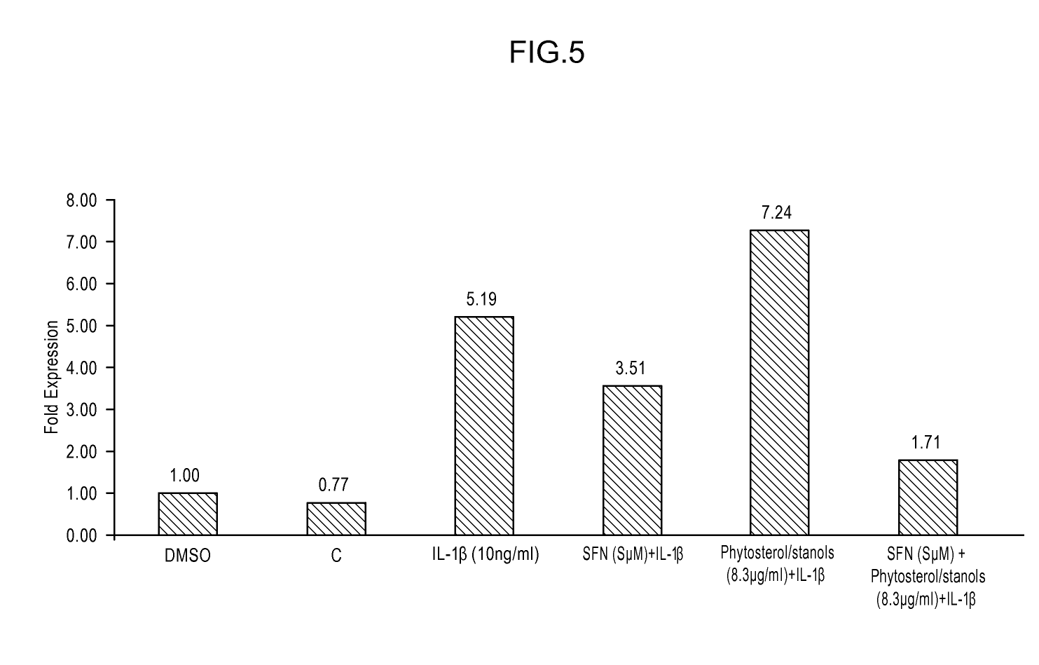

FIG. 6 is a graph showing the results of the experiment described in Example 7.

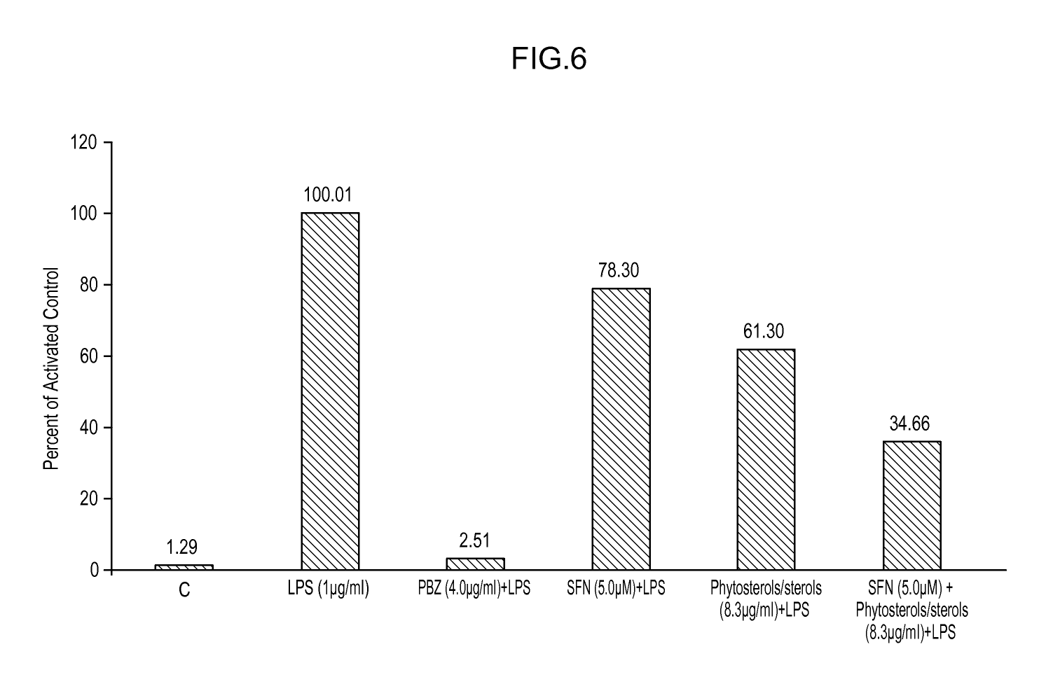

### DETAILED DESCRIPTION OF THE INVENTION

The present invention relates to the combination of a sulforaphane precursor, an enzyme capable of converting the sulforaphane precursor to sulforaphane, an enzyme potentiator, and a phytosterol and/or phytostanol or an ester thereof. The present invention also relates to the combination of sulforaphane or a derivative thereof and a phytosterol and/or phytostanol or an ester thereof. The present invention also relates to the combination of a broccoli extract or powder and a phytosterol and/or phytostanol or ester thereof, or mixtures thereof. The present invention also relates to the use a phytosterol and/or phytostanol or ester thereof, with a mixture of one or more of the following: sulforaphane precursor, sulforaphane or a derivative thereof, and broccoli extract. The present invention provides compositions relating to these combinations.

The present invention provides methods comprising administering these combinations. In some embodiments, the combination may be administered for treating, preventing, reducing the occurrence of, decreasing the symptoms associated with, and/or reducing secondary recurrences of, a disease or condition or damage associated with the connective tissue, liver, prostate, brain, spine, lung, kidneys, colon, breast, esophagus, pancreas, or ovaries in a subject. The combination may be administered for treating, preventing, reducing the occurrence of or degree of, decreasing the symptoms associated with inflammation in a subject. The combination may be administered for decreasing levels of or downregulatinq or decreasing gene expression of matrix metalloproteinases such as matrix metalloproteinase 13 (MMP-13) and/or prostaglandin E₂ (PGE₂) in a subject. The combination may also be administered for treating, preventing, reducing the occurrence of, decreasing the symptoms associated with, and/or reducing secondary recurrences of a condition or disorder associated with increased or abnormal levels of MMP-13 and/or PGE₂ in a subject. The combination may also be administered for inducing levels of glutathione which can be productive in minimizing or reducing the presence of harmful free radicals in the body, inhibiting or reducing any harmful effects of the iNOS/NO system, and decreasing pro-inflammatory gene expression.

Sulforaphane is also known as 1-isothiocyanato-4-methylsulfinylbutane. Derivatives of sulforaphane include, but are not limited to sulfoxythiocarbamate analogues of sulforaphane, 6-methylsulfinylhexyl isothiocyanate (6-HITC), and compounds which comprise the structure of sulforaphane with different side chains and/or various lengths of spacers between the isothiocyanato and sulfoxide groups. Examples of derivatives of sulforaphane include those described in the following references, each of which is incorporated herein by reference: Hu et al., Eur J Med Chem, 2013, 64:529-539; Ahn et al., Proc Natl Acad Sci USA, 2010, 107(21):9590-9595; and Morimistu et al., J. Biol. Chem. 2002, 277:3456-3463, and Baird et al., Arch Toxicol, 2011, 85(4):241-272.

In some embodiments, the composition comprises sulforaphane or a derivative thereof, preferably sulforaphane, in an amount of about 1 μg to about 10 g, preferably about 3 μg to about 5 g, preferably about 5 μg to about 1000 mg, preferably about 7 μg to about 750 mg, more preferably about 10 μg to about 500 mg, and most preferably about 100 μg to about 100 mg. In some embodiments, compositions suitable for human use comprise about 1 mg to about 20 mg.

In some embodiments, the methods of the present invention comprise administration of sulforaphane or a derivative thereof to a subject, preferably sulforaphane, in an amount of about 1 μg to about 10 g, preferably about 3 μg to about 5 g, preferably about 5 μg to about 1000 mg, preferably about 7 μg to about 750 mg, more preferably about 10 μg to about 500 mg, and most preferably about 100 μg to about 100 mg. In some embodiments wherein the subject is a human, the method comprises administration of about 1 mg to about 20 mg. In some embodiments, the methods of the present invention comprise administration of sulforaphane or a derivative thereof to a subject, preferably sulforaphane, in an amount of about 0.01 μg/kg to about 0.2 g/kg, preferably about 0.05 μg/kg to about 0.07 g/kg, more preferably about 0.07 μg/kg to about 15 mg/kg, more preferably about 0.1 μg/kg to about 11 mg/kg, and most preferably about 0.2 μg/kg to about 7 mg/kg. In some preferred embodiments wherein the subject is a human, the method comprises administration of about 2 μg/kg to about 2 mg/kg, alternatively about 0.01 mg/kg to about 1 mg/kg, alternatively about 0.1 mg/kg to about 0.4 mg/kg. The above amounts may refer to each dosage administration or a total daily dosage. The total daily dosage refers to the total amount of a compound or ingredient which is administered to a subject in a twenty-four hour period.

In some embodiments, the method comprises administration of more than one of a sulforaphane or a derivative thereof. In some embodiments, the compositions comprise more than one of a sulforaphane or a derivative thereof. For example, the methods or composition may comprise both sulforaphane and one or more derivatives thereof, or two or more derivatives. In some embodiments wherein the method or composition comprise more than one of a sulforaphane or a derivative thereof, the above amounts may refer to the amount of each sulforaphane or a derivative thereof, or the total amount of the more than one sulforaphane or derivative thereof.

The term “sulforaphane precursor” refers to any compound, substance or material which can be used to produce sulforaphane. In preferred embodiments, the sulforaphane precursor comprises a compound which can be converted or metabolized to sulforaphane, preferably by an enzyme. In some preferred embodiments, the sulforaphane precursor comprises glucoraphanin. Glucoraphanin is a glucosinolate which is also known as 4-methylsulfinylbutyl glucosinolate and 1-S-[(1E)-5-(methylsulfinyl)-N-(sulfonatooxy)pentanimidoyl]-1-thio-β-D-glucopyranose.

In some embodiments, the composition comprises about 1 μg to about 10 g, preferably about 250 μg to about 5 g, more preferably about 500 μg to about 2000 mg, even more preferably about 1 mg to about 750 mg, even more preferably about 1.5 mg to about 250 mg, even more preferably about 2 mg to about 100 mg, and most preferably about 3 mg to about 75 mg of the sulforaphane precursor, preferably glucoraphanin. In some embodiments, compositions suitable for human use comprise about 3.5 mg to about 50 mg of the sulforaphane precursor, preferably glucoraphanin.

In some embodiments, the method comprises administering the sulforaphane precursor, preferably glucoraphanin to a subject, in an amount of about 1 μg to about 10 g, preferably about 250 μg to about 5 g, more preferably about 500 μg to about 2000 mg, even more preferably about 1 mg to about 750 mg, even more preferably about 1.5 mg to about 250 mg, even more preferably about 2 mg to about 100 mg, and most preferably about 3 mg to about 75 mg. In some embodiments wherein the subject is a human, the method comprises administration of about 3.5 mg to about 50 mg. In some embodiments, the method comprises administering an amount of sulforaphane precursor to a subject in an amount of about 1 μg/kg to about 1000 mg/kg, preferably about 5 μg/kg to about 500 mg/kg, more preferably about 7.5 μg/kg to about 100 mg/kg, even more preferably about 10 μg/kg to about 25 mg/kg, and most preferably about 25 μg/kg to about 10 mg/kg. In some embodiments wherein the subject is a human, the method comprises administration of about 50 μg/kg to about 800 μg/kg. The above amounts may refer to each dosage administration or a total daily dosage.

In some embodiments, the method comprises administration of more than one sulforaphane precursor. In some embodiments, the composition comprises more than sulforaphane precursor. In some embodiments wherein the method or composition comprises more than one sulforaphane precursor, the above amounts may refer to the amount of each sulforaphane precursor, or the total amount of the sulforaphane precursors.

The sulforaphane precursor may be converted or metabolized to sulforaphane. In some embodiments, the sulforphane precursor is converted to sulforaphane by an enzyme. In some embodiments, the enzyme capable of converting the sulforaphane precursor to sulforaphane comprises a glucosidase enzyme, preferably a thioglucosidase enzyme, and more preferably myrosinase. Myrosinase is also known as thioglucoside glucohydrolase.

In some embodiments, the composition comprises the enzyme in an amount of about 1 pg to about 1 ug, preferably about 50 pg to about 500 ng, and most preferably about 1 ng to about 150 ng. In some embodiments, compositions suitable for human use comprise about 5 ng to about 75 ng of the enzyme.

In some embodiments, the method comprises administering the enzyme, preferably myrosinase, in an amount of about 1 pg to about 1 pg, preferably about 50 pg to about 500 ng, and most preferably about 1 ng to about 150 ng. In some embodiments wherein the subject is a human, the method comprises administration of about 5 ng to about 75 ng of the enzyme. In some embodiments, the method comprises administering the enzyme to a subject in an amount of about 0.02 pg/kg to about 0.02 ug/kg, preferably about 0.7 pg/kg to about 7 ng/kg, and most preferably about 0.02 ng/kg to about 2 ng/kg. In some preferred embodiments wherein the subject is a human, the method comprises administration of about 0.1 ng/kg to about 1 ng/kg. The above amounts may refer to each dosage administration or a total daily dosage.

In some embodiments, the method comprises administration of more than one enzyme capable of converting the sulforaphane precursor to sulforaphane. In some embodiments, the composition comprises more than one enzyme capable of converting the sulforaphane precursor to sulforaphane. In some embodiments wherein the methods or compositions comprise more than one enzyme, the above amounts may refer to the amount of each enzyme, or the total amount of the enzymes.

The present invention also provides for the use of a broccoli extract and/or powder, including but not limited to broccoli seed and sprout extracts and powders. The present invention provides methods of administration of broccoli extract and/or powder, and compositions comprising broccoli extract and/or powder. In some embodiments, the broccoli extract or powder is standardized to contain about 1% to about 75% w/w, more preferably about 2.5% to about 50%, even more preferably about 5% to about 25%, and most preferably about 10% to about 20% of a sulforaphane precursor, preferably glucoraphanin. Examples of broccoli extracts and powders include but are not limited to those described in U.S. Pat. Nos. 5,411,986; 5,725,895; 5,968,505; 5,968,567; 6,177,122; 6,242,018; 6,521,818; 7,303,770, and 8,124,135, each of which is incorporated by reference in its entirety. Powders of broccoli may be obtained, for example, by air drying, freeze drying, drum drying, spray drying, heat drying and/or partial vacuum drying broccoli, preferably broccoli sprouts. In some embodiments, the compositions and methods comprise use of about 1 μg to about 10 g, more preferably about 250 μg to about 5 g, even more preferably about 500 μg to about 1 g, preferably about 600 μg to about 500 mg, more preferably about 750 μg to about 400 mg, and most preferably about 1 mg to about 300 mg of the broccoli extract. In some embodiments, the broccoli extract or powder is present in a composition or administered to a subject in amounts sufficient to provide a sulforaphane precursor or sulforaphane in the amounts described above. In some embodiments, the composition may further comprise an enzyme potentiator, preferably ascorbic acid. In some embodiments, the method may further comprise administration of an enzyme potentiator, preferably ascorbic acid.

The sulforaphane or a derivative thereof, the sulforaphane precursor, and/or the enzyme capable of converting the sulforaphane precursor to sulforaphane may be obtained from any source, including but not limited to one or more plants from the Brassicaceae (also known as Cruciferae) family. Examples of plants from the Brassicaceae family include, but are not limited to, the following: broccoli, Brussels sprouts, cauliflower, cabbage, horseradish, parsnip, radish, wasabi, watercress, and white mustard. In some preferred embodiments, sulforaphane precursor, preferably glucoraphanin, and the enzyme, preferably myrosinase, are obtained from broccoli, broccoli sprouts, or broccoli seeds. The sulforaphane precursor and the enzyme may be obtained from the same source or from different sources. In some embodiments, both the sulforaphane precursor and the enzyme may be obtained from an extract or powder from these plants, preferably a broccoli seed or sprout extract or powder.

The present invention provides for the use of an enzyme potentiator. Enzyme potentiators may be used to enhance the activity of the enzyme that is capable of converting the sulforaphane precursor to sulforaphane. In some embodiments, the enzyme potentiator comprises an enzyme co-factor, preferably ascorbic acid. Ascorbic acid, also known as ascorbate or vitamin C, can potentiate the activity of myrosinase. In some embodiments, without an enzyme potentiator such as ascorbic acid, the conversion reaction to sulforaphane may be too slow to occur in the location needed for peak absorption. The enzyme potentiator may be obtained from a natural source, or it may be produced synthetically.

In some embodiments, the compositions may comprise about 1 mg to about 500 mg, preferably about 1 mg to about 250 mg, and most preferably about 1 mg to about 125 mg of the enzyme potentiator. In some preferred embodiments, compositions suitable for human use comprise about 1 mg to about 50 mg of the enzyme potentiator.

In some embodiments, the method of the present invention comprises administration of an enzyme potentiator, preferably ascorbic acid, in an amount of about 1 mg to about 500 mg, preferably about 1 mg to about 250 mg, and most preferably about 1 mg to about 125 mg. In some preferred embodiments wherein the subject is a human, the method comprises administration of about 1 mg to about 50 mg. In some embodiments, the method of the present invention comprises administration of the enzyme potentiator, preferably ascorbic acid, in an amount of about 0.01 mg/kg to about 3 mg/kg, and most about 0.02 mg/kg to about 2 mg/kg. In some preferred embodiments wherein the subject is a human, the method comprises administration of about 0.02 mg/kg to 0.7 mg/kg of the enzyme potentiator. The above amounts may refer to each dosage administration or a total daily dosage.

In some embodiments, the method comprises administration of more than one enzyme potentiator. In some embodiments, the composition comprises more than one an enzyme potentiator. In some embodiments wherein the method or composition comprise more than one enzyme potentiator, the above amounts may refer to the amount of each enzyme potentiator, or the total amount of the enzyme potentiators.

The present invention further comprises the use of a phytosterol or an ester thereof, and/or a phytostanol or an ester thereof, and/or mixtures thereof. The term “phytosterol” includes, but is not limited to, 4-desmethyl sterols, 4-monomethyl sterols, and 4,4-dimethyl sterols (triterpene alcohols) and mixtures thereof. Examples of 4-desmethyl sterols include, but are not limited to sitosterol, campesterol, stigmasterol, brassicasterol, 22-dehydrobrassicasterol, and 5-avenasterol. Examples of 4,4-dimethyl sterols include, but are not limited to cycloartenol, 24-methylenecycloartanol, and cyclobranol. The term “phytostanol” includes saturated forms of phytosterols including, but not limited to sitostanol, campestanol and their 24-epimers, and saturated forms of cycloartanol, 24-methylenecycloartanol, and cyclobranol, and mixtures thereof. The terms “phytosterol ester” and “phytostanol ester” refer to phytosterols and phytostanols which are esterified with acids, such as fatty acids. Examples of fatty acids include unsaturated and saturated fatty acids, including, but not limited to, myristoleic acid, palmitoleic acid, sapienic acid, oleic acid, elaidic acid, vaccenic acid, linoleic acid, linoelaidic acid, alpha-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, and cerotic acid. In some embodiments, examples of acids which can be esterified with phytosterol and phytostanols include, but are not limited to, palmitic acid, palmitoieic acid, oleic acid, linoleic acid, and stearic acid. In some embodiments, the present invention relates to the use of one phytosterol or an ester thereof, or one phytostanol or an ester thereof. In some embodiments, the present invention relates to the use of more than one phytosterols (or esters thereof) or the use of more than one phytostanols (or esters thereof). In some embodiments, the present invention relates to the use of a mixture of phytosterols and/or phytosterol esters, or a mixture of phytostanols and phytostanol esters, or a mixture of phytosterols and phytostanols and/or esters thereof. In some embodiments, the present invention provides for the use of sitosterol, campesterol, and/or stigmasterol, or a mixture thereof. In some embodiments, the present invention provides for the use of a mixture comprising sitosterol, campesterol, stigmasterol, campestanol, sitostanol, and brassicasterol.

The phytosterols, phytostanols, or esters thereof may be provided in any form, such as an extract or powder. In some embodiments, the extract or powder may comprise phytosterols, phytostanols or esters thereof which are isolated or extracted from vegetable oils such as soybean oil, rapeseed (canola) oil, safflower oil, cottonseed oil, sunflower oil, or corn oil, or from tall oil or tall oil pitch, as described in U.S. Pat. No. 8,338,564. Examples of extracts and powders include, but are not limited to Phytosterol Complex (marketed by Total Nutrition), Phytosterol Complex (marketed by Source Naturals), Heart Choice Plant Sterols (marketed by Vitamin Shoppe), and Phytosterol Complex (marketed by Puritan's Pride). In some embodiments, extract or powder comprises sitosterol, campesterol, and/or stigmasterol. In some embodiments, the extract or powder is standardized to contain about 5% to about 99%, alternatively about 15% to about 95%, alternatively about 30% to about 90%, or alternatively about 40% to about 80% of phytosterol, phytostanol, or an ester thereof. These percentage amounts may refer to a single phytosterol, phytostanol, or ester thereof, or the total amount of phytosterol, phytostanol, and esters thereof. The phytosterol and/or phytostanol-containing powders of the present invention may be obtained by any method in the art, including but not limited to air drying, freeze drying, drum drying, spray drying, heat drying and/or partial vacuum drying oil.

In some embodiments, the compositions and methods comprise use of about 1 mg to about 1000 mg of phytosterol, phytostanol, or an ester thereof. In some embodiments, the compositions and methods comprise use of about 5 mg to about 750 mg, alternatively about 10 mg to about 500 mg, alternatively about 15 mg to about 400 mg, alternatively about 20 mg to about 300 mg, alternatively about 25 mg to about 250 mg, alternatively about 25 mg to about 200 mg of phytosterol, phytostanol, or an ester thereof. These amounts may prefer to the amount of a single phytosterol, phytostanol, or ester thereof, or the total amount of phytosterol, phytostanol, and esters thereof. These amounts may also refer to the amount of a mixture, extract, or powder comprising a phytosterol, a phytostanol, or an ester thereof. In some embodiments, the compositions and methods comprise use of about 25 mg to about 200 mg of sitosterol, stigmasterol, and/or campesterol.

In some embodiments, the methods comprise administration of phytosterol, phytostanol, or ester thereof in an amount of about 0.01 mg/kg to about 15 mg/kg, alternatively about 0.05 mg/kg to about 10 mg/kg, alternatively about 0.1 mg/kg to about 8 mg/kg, alternatively about 0.2 mg/kg to about 6 mg/kg, alternatively about 0.35 mg/kg to about 5 mg/kg, or alternatively about 0.3 mg/kg to about 3 mg/kg. These amounts may prefer to the amount of a single phytosterol, phytostanol, or ester thereof, or the total amount of phytosterol, phytostanol, and esters thereof. These amounts may also refer to the amount of a mixture, extract, or powder comprising a phytosterol, a phytostanol, or an ester thereof. The above amounts may refer to each dosage administration or a total daily dosage.

The methods of the present invention may further comprise administration of one or more additional components. The compositions of the present invention may further comprise one or more additional components. The present invention also provides for methods and compositions comprising the use of one or more of these additional components, in addition to or in place of phytosterol, phytostanol, or ester thereof. A synergistic effect may be found with the use of the additional components. The additional components may include active pharmaceutical ingredients, nutritional supplements, and nutritional extracts. Examples of additional components include, but are not limited, quercetin or a derivative thereof, an aminosugar such as glucosamine, a glycosaminoglycan such as chondroitin, avocado/soybean unsaponifiables, vitamins such as vitamin K2, coffee fruit, magnesium, ursolic acid, proanthocyanidins, catechins, alpha- or beta-glucans, curcumin, S-adenosylmethionine (SAMe), betalains, lipoic acid, gallic acid, resveratrol, hyaluronic acid, boron, methylsulfonylmethane (MSM), and collagen type II. These additional components may be present in cranberry (Vaccinium macrocarpon) extract (proanthocyanidins, quercetin, and ursolic acid), turmeric (Curcuma longa), medicinal mushroom extract such as shiitake (Lentinus edodes), maitake (Grifola frondosa) mushroom extracts, milk thistle extract or powder, reishi (Ganoderma lucidum) mushroom extract, green tea extract, and egg shell membrane.

In some embodiments, the ratio of phytosterol, phytostanol, or ester thereof to sulforaphane or a derivative thereof is about 1:50 to about 1500:1, alternatively about 1:25 to about 1000:1, alternatively about 1:10 to about 750:1, alternatively about 1:5 to about 500:1, alternatively about 1:2 to about 250:1, alternatively about 2:1 to about 100:1, alternatively about 2:1 to about 50:1, alternatively about 2.5:1 to about 25:1, alternatively about 3:1 to about 15:1, alternatively about 3:1 to about 10:1, or alternatively about 3:1 to about 8:1. In some embodiments, the ratio of phytosterol, phytostanol, or ester thereof to sulforaphane precursor is about 1:50 to about 1000:1, alternatively about 1:25 to about 750:1, alternatively about 1:10 to about 500:1, alternatively about 1:5 to about 250:1, alternatively about 1:2 to about 150:1, alternatively about 2:1 to about 100:1, alternatively about 2.5:1 to about 75:1, alternatively about 3:1 to about 50:1, alternatively about 4:1 to about 25:1, alternatively about 4:1 to about 10:1, alternatively about 4:1 to about 7:1. These ratios may relate to the amount of one phytosterol or ester thereof, one phytostanol or ester thereof, or the total amount of phytosterol or ester thereof and phytostanol or ester thereof.

In some embodiments, the composition comprises a unit dosage form, including but not limited to pharmaceutical dosage forms suitable for oral, rectal, intravenous, subcutaneous, intramuscular, transdermal, transmucosal, and topical. In some preferred embodiments, the composition comprises an orally administrable dosage form or a rectally administrable dosage form. Examples of orally administrable dosage forms include, but are not limited to a tablet, capsule, powder that can be dispersed in a beverage, a liquid such as a solution, suspension, or emulsion, a soft gel/chew capsule, a chewable bar, or other convenient dosage form known in the art. In preferred embodiments, the composition comprises a tablet, capsule, or soft chewable treat. The orally administrable dosage forms may be formulated for immediate release, extended release or delayed release.

In some embodiments, at least the sulforaphane precursor, the enzyme, and the enzyme potentiator are provided in a dosage form which allows for the release in an area of the gastrointestinal tract having a pH of at least 4 and preferably at least 5, such as the small intestine, preferably the duodenum. In some embodiments, at least the sulforaphane or derivative thereof and/or the broccoli extract or powder are provided in a dosage form which allows for the release in an area of the gastrointestinal tract having a pH of at least 4 and preferably at least 5, such as the small intestine, preferably the duodenum. In some embodiments, the phytosterol and/or phytostanol or ester thereof (or a mixture thereof) and/or any optional additional components are also released in an area of the gastrointestinal tract having a pH of at least 4 and preferably at least 5, such as the small intestine, preferably the duodenum. The small intestine includes the duodenum, jejunum, and ileum.

In some embodiments, each of these components (i.e, sulforaphane precursor, enzyme, enzyme potentiator, sulforaphane or a derivative thereof, broccoli extract or powder, phytosterol and/or phytostanol or ester thereof (or a mixture thereof), and/or additional components) are released simultaneously or concomitantly (i.e., within a short period of time of each other). This provides benefits over glucoraphanin-containing compositions formulated to release the glucoraphanin in an area of the gastrointestinal tract having a pH below 4, such as the stomach. In low pH environments such as this, the acidic environment may divert conversion of sulforaphane precursor to other, physiologically inactive end products, such as sulforaphane nitrile and epithionitrile.

In some embodiments, the compositions may comprise orally administrable compositions which comprise gastroprotective formulations, including enteric coated dosage forms or any dosage form which is resistant to degradation in an area of the gastrointestinal tract having pH below 4, such as the stomach. For example, the orally administrable composition may comprise a tablet or capsule comprising an enteric coating. The enteric coating may comprise materials including, but not limited to cellulose acetate phthalate, hydroxypropyl methylcellulose phthalate, polyvinyl acetate phthalate, methacrylic acid copolymer, methacrylic acid:acrylic ester copolymer, hydroxypropyl methylcellulose acetate succinate, hydroxypropyl methylcellulose trimellitate, shellac, cellulose acetate trimellitate, carboxymethylethylcellulose, and mixtures thereof. The enteric coating may comprise any suitable enteric polymers known in the art. In some embodiments, one or more of the components in the composition may be embedded in a matrix of enteric polymers. In some embodiments, the orally administrable compositions comprise a capsule that dissolves slowly in gastric acid and travels to the small intestine, such as DRCAPS™ acid resistant capsules, which are marketed by CAPSUGEL® or any other acid resistant capsules.

In the most preferred form, the orally administrable composition is surrounded by a coating that does not dissolve unless the surrounding medium is at a pH of at least 4, and more preferably at least 5. Alternatively, a coating may be employed which controls the release by time, as opposed to pH, with the rate adjusted so that the components are not released until after the pH of the gastrointestinal tract has risen to at least 4, and more preferably at least 5. Thus, a time-release formulation may be used to prevent gastric presence of the sulforaphane precursor, the enzyme capable of converting the sulforaphane precursor to sulforaphane, and the enzyme potentiator, or of the sulforaphane. The coating layer(s) may be applied onto orally administrable composition using standard coating techniques. The enteric coating materials may be dissolved or dispersed in organic or aqueous solvents. The pH at which the enteric coat will dissolve can be controlled by a polymer, or combination of polymers, selected and/or ratio of pendant groups. For example, dissolution characteristics of the polymer film can be altered by the ratio of free carboxyl groups to ester groups. Enteric coating layers also contain pharmaceutically acceptable plasticizers such as triethyl citrate, dibutyl phthalate, triacetin, polyethylene glycols, polysorbates or other plasticizers. Additives such as dispersants, colorants, anti-adhering and anti-foaming agents may also be included.

The compositions may contain one or more non-active pharmaceutical ingredients (also known generally as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. The excipients are preferably pharmaceutically acceptable excipients. Examples of classes of pharmaceutically acceptable excipients include lubricants, buffering agents, stabilizers, blowing agents, pigments, coloring agents, flavoring agents, fillers, bulking agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof.

In some embodiments, the combination of (i) a sulforaphane precursor, preferably glucoraphanin, (ii) an enzyme capable of converting the sulforaphane precursor to sulforaphane, preferably a glucosidase enzyme, more preferably a thioglucosidase enzyme, and most preferably myrosinase, (iii) an enzyme potentiator, preferably an enzyme co-factor, more preferably ascorbic acid, and (iv) phytosterol and/or phytostanol or ester thereof (or a mixture thereof) demonstrates a synergistic effect. In some embodiments, the combination of sulforaphane (or a derivative thereof) and a phytosterol, a phytostanol, or ester thereof (or a mixture thereof) demonstrates a synergistic effect. Synergy refers to the effect wherein a combination of two or more components provides a result which is greater than the sum of the effects produced by the agents when used alone. In preferred embodiments, the synergistic effect is greater than an additive effect. In some embodiments, the combination of a sulforaphane precursor, an enzyme capable of converting the sulforaphane precursor to sulforaphane, an enzyme potentiator, and a phytosterol, a phytostanol or ester thereof (or a mixture thereof) has a statistically significant, greater effect compared to: (i) each component alone, (ii) the combination of sulforaphane precursor and the enzyme alone; and/or (iii) the combination of sulforaphane precursor, the enzyme, and the enzyme potentiator alone.

In preferred embodiments, the combination of the sulforaphane precursor, the enzyme, the enzyme potentiator, and a phytosterol, a phytostanol, or ester thereof (or a mixture thereof) demonstrates synergy by having a statistically significant and/or greater than additive effect compared to the sulforaphane precursor alone and the phytosterol, phytostanol or ester thereof (or a mixture thereof) alone. In some embodiments, the combination of glucoraphanin, myrosinase, ascorbic acid, and phytosterol, phytostanol, or ester thereof (or a mixture thereof) has a synergistic effect compared to the combination of glucoraphanin, myrosinase, ascorbic acid alone; and compared to the phytosterol, phytostanol, or ester thereof (or a mixture thereof) alone. In some embodiments, the combination of glucoraphanin, myrosinase, ascorbic acid, and a mixture of one or more phytosterols, phytostanols, or esters thereof has a synergistic effect compared to the combination of glucoraphanin, myrosinase, ascorbic acid alone; and compared to a single phytosterol, phytostanol, or ester thereof.

In preferred embodiments, the combination of the sulforaphane (or a derivative thereof) and a phytosterol, a phytostanol, or ester thereof (or a mixture thereof) demonstrates synergy by having a statistically significant and/or greater than additive effect compared to the sulforaphane (or derivative thereof) alone and the phytosterol, phytostanol or ester thereof (or a mixture thereof) alone. In some embodiments, the combination of sulforaphane (or a derivative thereof), and a mixture of one or more phytosterols, phytostanols, or esters thereof has a synergistic effect compared to the combination of sulforaphane (or a derivative thereof); and compared to a single phytosterol, phytostanol, or ester thereof alone.

In some embodiments, the combination of broccoli extract or powder and a phytosterol, a phytostanol, or an ester thereof (or a mixture thereof) has a statistically significant and/or greater than additive effect than: (i) broccoli extract or powder alone, and/or (ii) a phytosterol, phytostanol, or ester thereof (or a mixture thereof) alone. In some embodiments, the combination of broccoli extract or powder and phytosterol and/or phytostanol or ester thereof (or a mixture thereof) has a synergistic effect compared to broccoli extract or powder alone, and a phytosterol, phytostanol, or ester thereof (or a mixture thereof) alone. In some embodiments, the combination of broccoli extract or powder and a mixture of one or more phytosterols, phytostanols, or esters thereof has a synergistic effect compared to the broccoli extract or powder alone; and compared to a single phytosterol, phytostanol, or ester thereof.

In some embodiments, the methods and compositions further comprise use of Boswellia (Boswellia serrata) extract or any components found in Boswellia extract, including but not limited to boswellic acid and pentacyclic triterpene acids. Examples of components include, but are not limited, to α-boswellic acid, β-boswellic acid, 3-acetyl α-boswellic acid, 3-acetyl β-boswellic acid, 11-keto-β-boswellic acid (KBA) and acetyl-11-keto-β-boswellic acid (AKBA). In some embodiments, the addition of Boswellia extract and/or components of Boswellia extract to the combinations of the present invention may have a synergistic effect compared to the combination alone.

The present invention provides methods of use, including methods of administration to a subject in need thereof. In some embodiments, the method comprises administration of the combination of a sulforaphane precursor, an enzyme capable of converting the sulforaphane precursor to sulforaphane, an enzyme potentiator, and a phytosterol, phytostanol, or ester thereof (or a mixture thereof). In some embodiments, the method comprises administration of the combination of a sulforaphane or a derivative thereof and a phytosterol, phytostanol, or ester thereof (or a mixture thereof). In some embodiments, the method comprises administration of the combination of a broccoli extract or powder and a phytosterol, phytostanol, or ester thereof (or a mixture thereof).

In some embodiments, the method relates to treating, preventing, reducing the occurrence of, decreasing the symptoms associated with, and/or reducing secondary recurrences of, a disease or condition associated with the connective tissue, liver, genitourinary system (including prostate, breast, and ovaries), brain, lung, kidneys, colon, esophagus, pancreas, or hematopoietic system in a subject, comprising administering to the subject. The methods may be useful in reducing damage of slowing damage to tissues and organs, such as the connective tissue, liver, genitourinary system (including prostate, breast, and ovaries), brain, lung, kidneys, colon, esophagus, and pancreas. In some embodiments, the method relates to increasing glutathione levels in a subject in need thereof in a subject. The method may also be useful in treating, preventing, decreasing the symptoms associated with, and/or reducing secondary recurrences of diseases or conditions associated with abnormal or elevated levels of pro-inflammatory mediators, such as matrix metalloproteinase-13 (MMP-13) and prostaglandin E₂ (PGE₂). Examples of such diseases and conditions include, but are not limited to, osteoarthritis, rheumatoid arthritis, non-alcoholic fatty liver disease (NAFLD), cancer (such as cancer of the liver, lung, prostate, colon, breast, brain, ovaries, esophagus, pancreas, nasopharynx, osteosarcoma), leukemia, cystic fibrosis, HIV, glutathione synthetase deficiency, cognitive dysfunction, Alzheimer's disease, Parkinson's disease, Huntington's disease, amyotrophic lateral sclerosis, Friedreich's ataxia, multiple sclerosis, fibromyalgia, chronic fatigue, autism, diabetes, hepatotoxicity, and toxicity due to environmental factors.

In some embodiments, the methods relate to providing a beneficial effect on biomarkers, and treating, preventing, reducing the occurrence of, decreasing the symptoms associated with abnormal levels of these biomarkers. Examples of such biomarkers include, but are not limited to NADPH-dependent enzymes, thioredoxin (TXN), thioredoxin reductase-1 (Txnrd-1), glutamate-cysteine ligase subunit (GCLC), sulfotransferase 1A1 (SULT1A1), heme oxygenase-1 (HMOX1), glutathione peroxidase-3 (GPx-3), glutathione S-transferase theta 2 (GSTT2), microsomal glutathione S-transferase 1 (MGST1), aldehyde oxidase (AOX1), aldo-keto reductase 1B8 (Akr1 b8), flavin-containing monooxygenase 2 (FMO2), Fc receptor region receptor III (Fcgr3), tryptase beta 1 (TPSB1), mast cell protease-6 (Mcpt6), neurexin-1-alpha (NRXN-1), microphthalmia-associated transcription factor (MITF), type II iodothyronine deiodinase (DIO2), angiopoietin-14 (Angpt14), cluster of differentiation (CD36), and Ntel. Diseases or conditions associated with elevated or abnormal levels of these biomarkers include, but are not limited to cancer, pulmonary and central nervous system tuberculosis, multiple sclerosis, Crohn's disease, atherosclerosis, osteoarthritis, asthma, stroke, emphysema, diabetic nephropathy, chronic histiocytic intervillositis of the placenta, hypertension, abdominal aortic aneurysm, inflammatory bowel disease, chronic rhinosinusitis, coronary artery disease, and kidney disease.

In some embodiments, the method comprises administering to a subject in need thereof a combination of sulforaphane and a phytosterol and/or phytostanol or ester thereof (or a mixture thereof). In some embodiments the method comprises administering to a subject in need thereof a combination of broccoli extract or powder and a phytosterol and/or phytostanol or ester thereof (or a mixture thereof). In some preferred embodiments, the method comprises administering to the subject a combination of glucoraphanin, myrosinase, ascorbic acid, and a phytosterol and/or phytostanol or ester thereof (or a mixture thereof). In preferred embodiments, the combinations demonstrate a synergistic effect in the methods of the present invention.

In preferred embodiments, one or more components of the combinations (for example, the sulforaphane precursor, the enzyme capable of converting the sulforaphane precursor to sulforaphane, the enzyme potentiator, the a phytosterol and/or phytostanol or ester thereof (or a mixture thereof); or the sulforaphane or derivative thereof and the phytosterol, phytostanol, or ester thereof (or a mixture thereof); or the broccoli extract or powder and the phytosterol and/or phytostanol or ester thereof (or a mixture thereof) are administered together in one composition or dosage form, or separately, preferably within a period in which their therapeutic properties overlap. In some embodiments, the components of the combinations may be administered in two or more orally administrable compositions or dosage forms. For example, in some embodiments, the sulforaphane precursor, the enzyme capable of converting the sulforaphane precursor to sulforaphane, and the enzyme potentiator are administered in one orally administrable dosage form, while the phytosterol, phytostanol, or ester thereof (or a mixture thereof) are administered in one or more separate or additional orally administrable dosage form(s). In preferred embodiments, the components of the combination are administered in one dosage form.

In some embodiments, the combination may be administered at a frequency of 1 to 10 times daily, preferably 1 to 5 times daily, more preferably 1 to 3 times daily, and most preferably 1 time daily.

The dosages disclosed in this application refer generally to dosages suitable for humans (approximately 68 kg). Dosage calculations can be determined by those of skilled in the art by evaluating body weight, surface area, metabolic rate, and species differences.

The term “subject” refers to any animal, including mammals and birds. Mammals include, but are not limited to, humans, dogs, cats, horses, cows, camels, elephants, lions, tigers, bears, seals, and rabbits. In preferred embodiments, the subjects comprise mammals that are not consumed as food, such as humans, cats, and dogs.

### EXAMPLES

### Example 1

The following is an exemplary formulation:

Glucoraphanin-containing broccoli extract (about 12% w/w), 50 mg to 5 g

Myrosinase-containing freeze-dried broccoli sprout powder, 25 mg to 500 mg

Ascorbic acid, 5 mg to 500 mg

Tall oil phytosterols and phytostanols, 25 to 50 mg

### Example 2

A Hydrophobic Interaction Chromatographic (HILIC) method was developed, comprising the following conditions:

Column: Waters BEH Amide, 1.7-μm particle size; 2.1 mm×100 mm

Mobile Phase: 20% 10 mM Ammonium Acetate, pH 5.0; 80% Acetonitrile;

Separation mode: isocratic

Column Temperature: 70° C.

Flow Rate: 0.7 mL/min

The above conditions allow separation of five typical Brassicaceae glucosinolates, including the sulforaphane precursor, glucoraphanin.

### Example 3

### Consumption of Glucoraphanin as a Function of the Ascorbic Acid Concentration

About 250 mg of broccoli seed extract containing about 12% (w/w) glucoraphanin were subjected to hydrolysis by a fixed concentration of broccoli sprout-derived myrosinase in the presence of variable concentration of ascorbic acid, ranging from 0 to 600 μmoles/Liter. The reaction mixtures were thermostated at 38° C.; aliquots were withdrawn every 15 minutes for 60 minutes, and concentration of glucoraphanin determined chromatographically. The rate of glucoraphanin consumption was interpreted as the rate its conversion to sulforaphane. Graphical representation of glucoraphanin content reduction as a function of increasing ascorbic acid concentration results in a series of linear plots; the slopes of the linear regression lines reflect the rate of glucoraphanin consumption, in μmoles/minute. It is apparent that in the presence of 600 μmoles/Liter concentration of ascorbic acid, the reaction rate increased 13-fold relative to that which proceeded in the absence of modulatory effects of ascorbic acid.

| | Content of Ascorbic Acid | | | | | | | |
|---|---|---|---|---|---|---|---|---|
| | | | | | 250 μM | | | |
| Time, min | 0 μM | 50 μM | 125 μM | 250 μM | Filtered | 400 μM | 600 μM | |
| 0 | 93.36 | 93.36 | 93.36 | 93.36 | 93.36 | 93.36 | 93.36 | μmoles |
| 15 | 92.24 | 89.20 | 84.52 | 80.95 | 86.31 | 78.32 | 75.02 | GR |
| 30 | 90.71 | 84.24 | 75.92 | 69.06 | 79.44 | 62.78 | 55.66 | |
| 45 | 89.44 | 80.30 | 68.09 | 57.63 | 71.94 | 47.67 | 37.50 | |
| 60 | 87.79 | 76.36 | 59.41 | 45.76 | 65.18 | 33.15 | 22.09 | |
| Slope | −0.09293 | −0.28599 | −0.56217 | −0.79012 | −0.47140 | −1.00714 | −1.20029 | μmol/min |
| Intercept | 93.496 | 93.271 | 93.123 | 93.053 | 93.386 | 93.270 | 92.734 | μmol |

### Example 4

### Equimolar Conversion of Glucoraphanin to Sulforaphane

A two-part experiment was conducted to further elucidate the role of ascorbic acid in modulating myrosinase activity. All solutions were prepared in 20 mM Tris-buffered saline, at pH 7.5, previously identified as an optimal for myrosinase activity; each sample tube had 100 mg of freeze-dried broccoli powder accurately weighed in as a source of myrosinase. Experiment was conducted at 38° C. for 2 hours, with sample aliquots removed in 30-minute increments, and both glucoraphanin and sulforaphane content assessed by HPLC. A strongly acidic “stop” solution was utilized to instantaneously inhibit further myrosinase activity in the removed aliquots. A control sample contained no ascorbic acid, and the enzymatic conversion proceeded unassisted by a co-factor.

PART 1. In the presence of the fixed concentration of ascorbic acid, 1 mmol/Liter, an increasing amount of broccoli seed extract (about 12% glucoraphanin, w/w) was added, ranging from 250 mg to 500 mg.

PART 2. While keeping the amount of broccoli seed extract fixed at 250 mg, the concentration of ascorbic acid was varied from 0.4 mmol/Liter to 3.8 mmol/Liter. The table below presents glucoraphanin and sulforaphane expressed in μmoles. It is apparent that within the first 30 minutes in almost all the reaction mixtures, conversion of glucoraphanin to sulforaphane was complete. However, careful examination of the enzymatic conversion occurring in the control sample, without the stimulating effects of ascorbic acid, reveals an equimolar conversion of glucoraphanin to sulforaphane, i.e., the amount of glucoraphanin consumed results in the equivalent amount of sulforaphane produced.

| | Glucoraphanin, μmoles | | | | | Sulforaphane, μmoles | | | | |
|---|---|---|---|---|---|---|---|---|---|---|
| | Time, min | | | | | | | | | |
| | 0 | 30 | 60 | 90 | 120 | 0 | 30 | 60 | 90 | 120 |
| GR 250 mg AA 0.0 mM | 58.02 | 48.57 | 37.52 | 26.58 | 15.67 | 3.42 | 12.08 | 22.27 | 33.17 | 42.89 |
| GR 250 mg AA 1.0 mM | 40.07 | | | | | 21.51 | 61.95 | 60.20 | 60.04 | 58.25 |
| GR 300 mg AA 1.0 mM | 49.31 | | | | | 24.18 | 74.40 | 73.04 | 72.19 | 70.56 |
| GR 350 mg AA 1.0 mM | 61.41 | | | | | 25.00 | 84.92 | 84.02 | 83.19 | 80.02 |
| GR 400 mg AA 1.0 mM | 71.35 | 1.56 | | | | 26.71 | 96.60 | 95.38 | 93.39 | 91.16 |
| GR 500 mg AA 1.0 mM | 89.41 | 1.01 | | | | 33.52 | 120.16 | 118.45 | 116.45 | 112.34 |
| GR 250 mg AA 0.4 mM | 45.66 | | | | | 15.98 | 62.06 | 61.01 | 60.88 | 58.90 |
| GR 250 mg AA 1.0 mM | 35.24 | | | | | 26.49 | 62.19 | 60.62 | 60.41 | 59.10 |
| GR 250 mg AA 2.0 mM | 24.94 | | | | | 36.05 | 60.85 | 59.78 | 59.65 | 58.08 |
| GR 250 mg AA 2.9 mM | 22.24 | | | | | 38.20 | 59.95 | 59.34 | 58.77 | 56.99 |
| GR 250 mg AA 3.8 mM | 21.70 | | | | | 37.87 | 58.77 | 57.79 | 58.41 | 56.17 |

In the Part 2 of the experiment, the modulatory effect of the increasing concentration of ascorbic acid on the activity of myrosinase was assessed. An initial, apparently linear, increase in myrosinase-promoted conversion of glucoraphanin to sulforaphane is observed to about 2 mmol/L of ascorbic acid concentration, followed subsequently by a considerable leveling off.

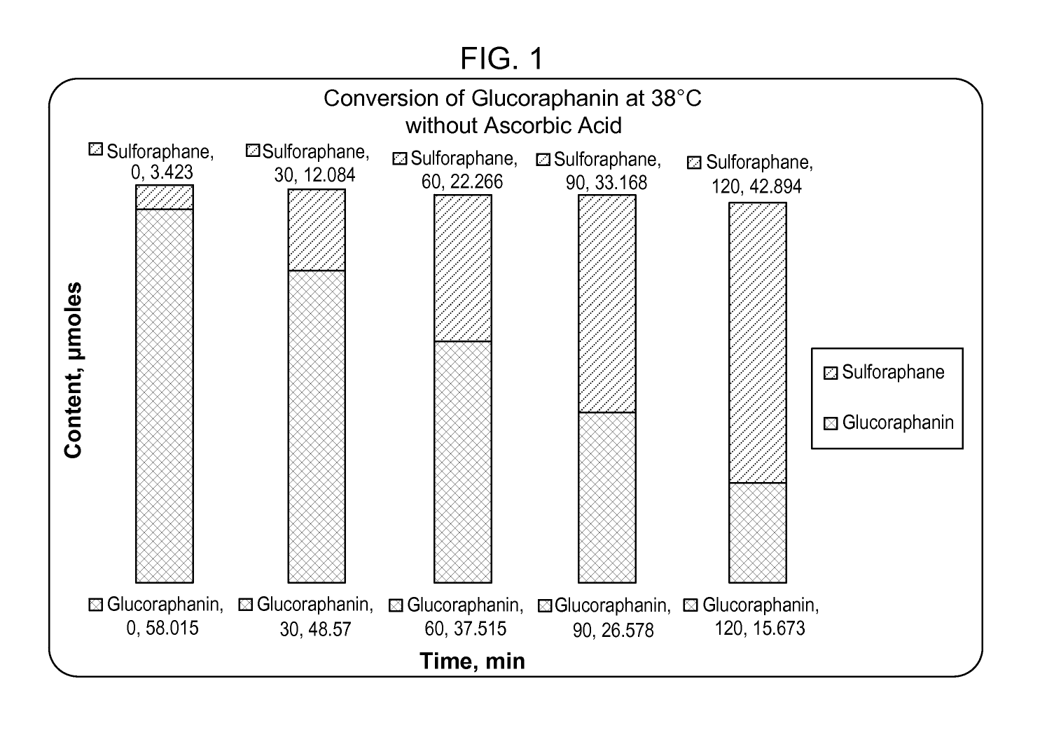

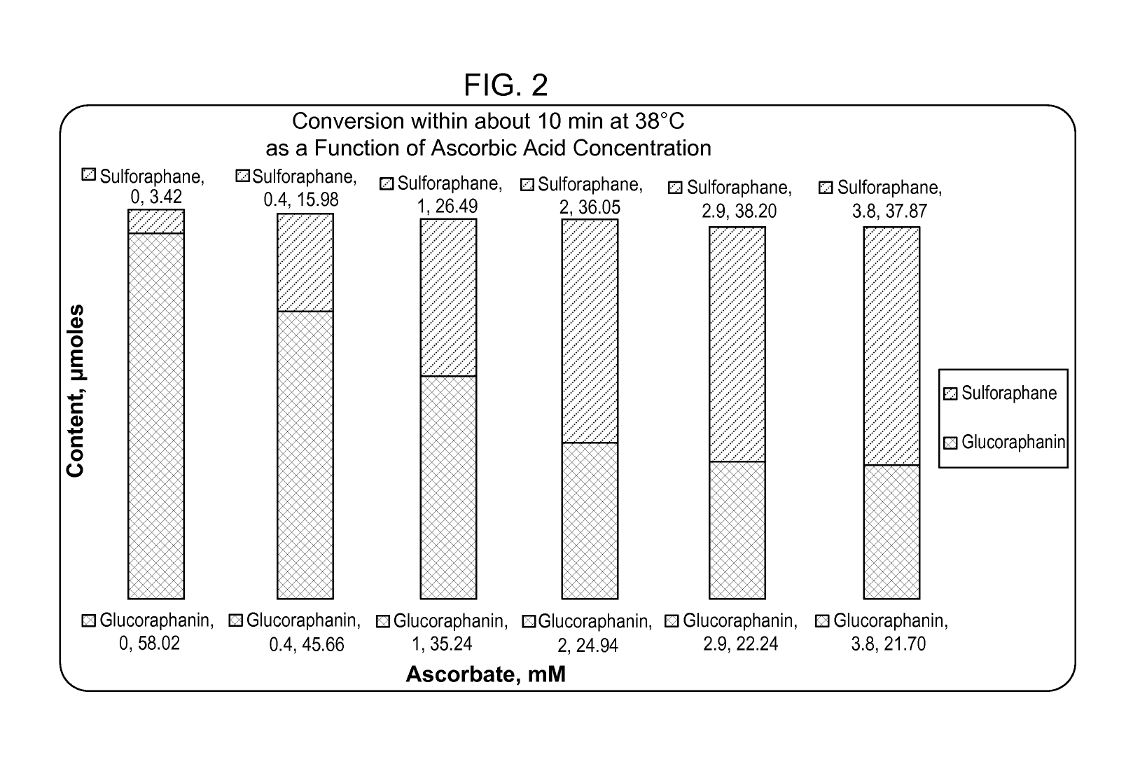

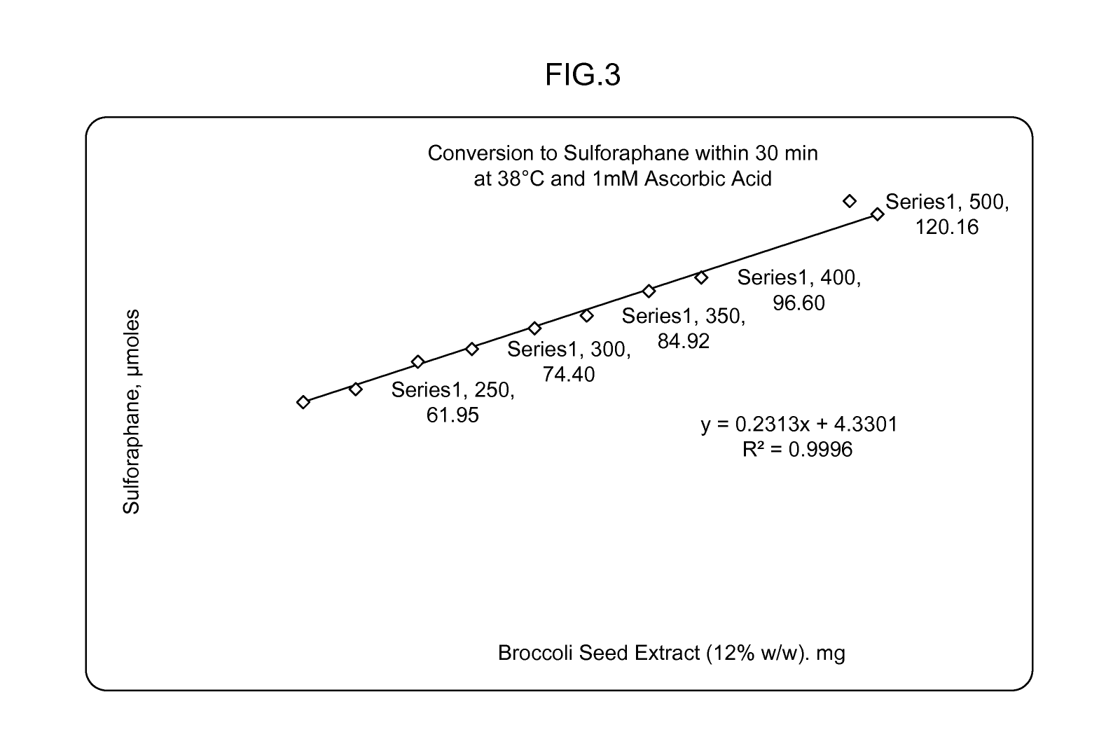

Finally, examination of sulforaphane yield of after 30 minutes within the PART 1 of the experiment, reveals that in the presence of 1 mmol/Liter of ascorbic acid, the fixed amount of myrosinase contained in 100 mg of freeze-dried broccoli sprout powder is capable of generating at least 200 μmoles of sulforaphane, in a predictably linear fashion. FIGS. 1, 2, 3, and 4 demonstrate the results of this study.

### Example 5

### Conversion of Glucoraphanin to Sulforaphane in the Presence of Simulated Intestinal Fluid

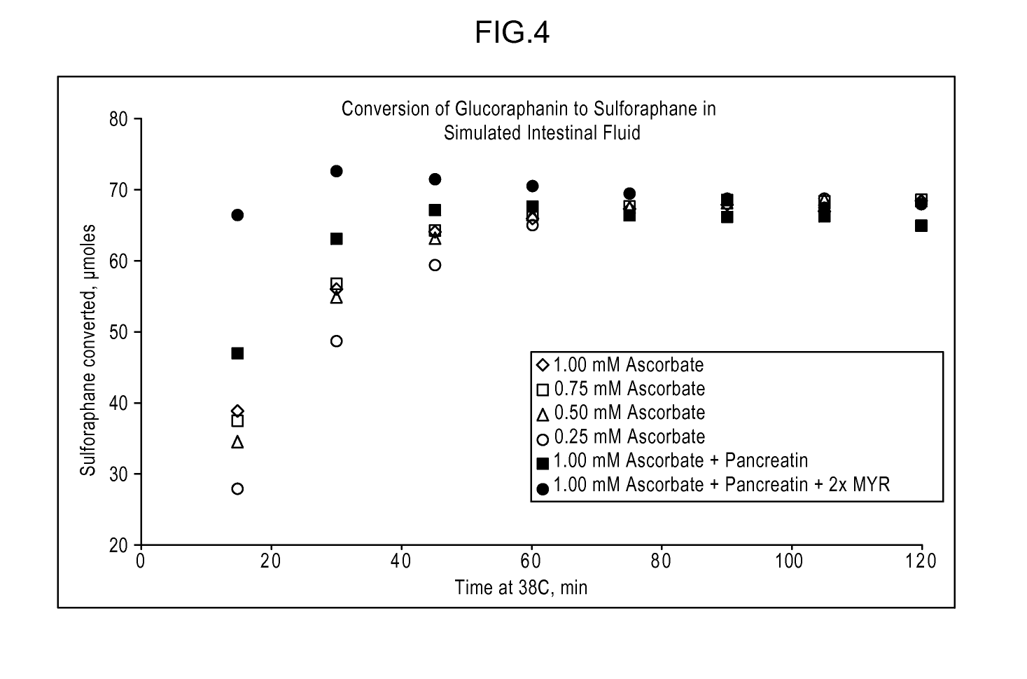

Simulated Intestinal Fluid (SIF) powder, a commercially supplied concentrate closely approximating the human intestinal content in terms of composition, pH and ionic strength, was used. The experiment utilized a USP Dissolution Apparatus 2 (paddles), where into six dissolution vessels 500 mL of Simulated Intestinal Fluid was dispensed, along with 150 mg of freeze-dried broccoli sprout powder as a source of myrosinase. In vessels 1-4, the concentration of ascorbic acid was varied from 0.25 to 1.00 mmol/Liter; in vessel 5, in addition to 1 mmol/Liter ascorbic acid, 3.125 g of pancreatin (8×USP) was suspended; in vessel 6, in addition to 1 mmol/Liter ascorbic acid, and 3.125 g of pancreatin (8×USP), a doubled amount of freeze-dried broccoli sprout powder (300 mg) was added. After vessels were brought to 38° C., 250 mg of glucoraphanin-rich (12%, w/w) broccoli seed extract was added to each, and the resulting suspensions were stirred at 75 RPM for 2 hours. Aliquots were withdrawn every 15 minutes, and assayed for sulforaphane. FIG. 4 shows direct correlation between larger yield of sulforaphane and higher concentrations of ascorbic acid, especially at the earlier stages of the experiment.

### Example 6

The following study was conducted to determine the effect of the combination of phytosterols on levels of gene expression of matrix metalloproteinase 13 (MMP-13). MMP-13 is a major type II collagen-degrading collagenase that is often used as a marker for progression of inflammatory disorders such as osteoarthritis. MMP-13 is regulated by both stress and inflammatory signals. Downregulation of MMP-13 expression is beneficial for joint health.

In the study, equine chondrocytes were treated with either: (1) 0.5 μM sulforaphane (SFN), (2) 8.3 μg/mL of a mixture of phytosterols and phytostanols, or (3) the combination of 0.5 μM sulforaphane (SFN) and 8.3 μg/mL of a mixture of phytosterols and phystostanols for 24 hours. Following pre-treatment, the chondrocytes were activated by interleukin-1β (IL-1β) for 24 hours to induce gene expression of MMP-13, which encodes a protein responsible for breaking down the extracellular matrix or support system of cells. MMP-13 levels were assessed via quantitative RT-PCR and presented as fold expression.

The results demonstrate that the combination of sulforaphane and MMP-13 had a synergistic effect, compared to each alone. In fact, the results show that the phytosterols and phytostanol mixture alone resulted in an increase in gene expression. However, the combination of sulforaphane and the phytosterols and phytostanols synergistically decreased MMP-13 gene expression.

### Example 7

The following study was conducted to determine the effect of the combination of phytosterols and phytostanols on levels of prostaglandin E₂ (PGE₂) production. PGE₂ is a pain and pro-inflammatory mediator which is often found in inflamed tissue. PGE₂ is thought to cause pain by directly exciting nociceptive primary sensory neurons (also called nociceptors) and indirectly stimulating the release of pain-related peptide substance P (SP) and calcitonin gene-related peptide (CGRP).

In the study, RAW mouse macrophage cells were treated with either: (1) 0.5 μM sulforaphane (SFN), (2) 8.3 μg/mL of a mixture of phytosterols and phytostanols or (3) the combination of 0.5 μM sulforaphane (SFN) and 8.3 μg/mL of phytosterols and phytostanols for 24 hours. The cells were then activated with LPS to induce inflammation and the production of PGE₂. The production of PGE₂ was assessed via ELISA. The results show that the combination of sulforaphane and phytosterols and phystanols resulted in a synergistic effect, compared to each alone. The combination resulted in a decrease of PGE₂ production.

1. An orally administrable composition comprising:
a sulforaphane precursor;
an enzyme capable of converting the sulforaphane precursor to sulforaphane;
an enzyme potentiator; and
a phytosterol and/or phytostanol or ester thereof.

2. The orally administrable composition of claim 1, wherein the sulforaphane precursor comprises glucoraphanin.

3. The orally administrable composition of claim 1, wherein the enzyme capable of converting the sulforaphane precursor to sulforaphane comprises myrosinase.

4. The orally administrable composition of claim 1, wherein the enzyme potentiator comprises ascorbic acid.

5. The orally administrable composition of claim 1, wherein the composition comprises an enteric-coated dosage form.

6. The orally administrable composition of claim 1, wherein the composition further comprises one or more additional components selected from the group consisting of: quercetin, an aminosugar, a glycosaminoglycan, avocado/soybean unsaponifiables, a vitamin, coffee fruit, magnesium, ursolic acid, a proanthocyanidin, a catechin, an alpha- or beta-glucan, curcumin, S-adenosylmethionine (SAMe), betalains, lipoic acid, gallic acid, resveratrol, hyaluronic acid, boron, methylsulfonylmethane (MSM), acetyl-keto-beta-boswellic acid (AKBA), and collagen type II.

7. The orally administrable composition of claim 1, comprising glucoraphanin, myrosinase, ascorbic acid, and a mixture comprising one or more phytosterols and/or phytostanols.

8. The orally administrable composition of claim 1, wherein the composition comprises a broccoli extract or powder comprising the sulforaphane precursor.

9. A method of treating, preventing, reducing the occurrence of, decreasing the symptoms associated with, and reducing secondary recurrences of a condition or disorder associated with connective tissue, comprising administering to a subject in need thereof a sulforaphane precursor; an enzyme capable of converting the sulforaphane precursor to sulforaphane; an enzyme potentiator; and a phytosterol and/or phytostanol or ester thereof.

10. The method of claim 9, wherein the sulforaphane precursor comprises glucoraphanin.

11. The method of claim 9, wherein the enzyme capable of converting the sulforaphane precursor to sulforaphane comprises myrosinase.

12. The method of claim 9, wherein the enzyme potentiator comprises ascorbic acid.

13. The method of claim 9, comprising administration of glucoraphanin, myrosinase, ascorbic acid, and a mixture comprising one or more phytosterols and/or phytostanols.

14. The method of claim 9, comprising administering an enteric-coated dosage form.

15. An orally administrable composition comprising:
sulforaphane or a derivative thereof; and
a phytosterol and/or phytostanol or ester thereof.

16. The orally administrable composition of claim 15, wherein the composition comprises an enteric-coated dosage form.

17. The orally administrable composition of claim 15, further comprising one or more additional components selected from the group consisting of: quercetin, an aminosugar, a glycosaminoglycan, avocado/soybean unsaponifiables, a vitamin, coffee fruit, magnesium, ursolic acid, a proanthocyanidin, a catechin, an alpha- or beta-glucan, curcumin, S-adenosylmethionine (SAMe), betalains, lipoic acid, gallic acid, resveratrol, hyaluronic acid, boron, methylsulfonylmethane (MSM), acetyl-keto-beta-boswellic acid (AKBA), and collagen type II.

18. The orally administrable composition of claim 15, wherein the composition comprises a broccoli extract or powder comprising the sulforaphane or a derivative thereof.

19. The orally administrable composition of claim 15, comprising the phytosterol and/or phytostanol or ester thereof and the sulforaphane or a derivative thereof provided in a ratio of from about 1:50 to about 1500:1.

20. The orally administrable composition of claim 19, comprising the phytosterol and/or phytostanol or ester thereof and the sulforaphane or a derivative thereof provided in a ratio of from about 3:1 to about 8:1.
